(19) United States
(12) Patent Application Publication
(10) Pub. No.: US 2021/0022619 A1
(43) Pub. Date: Jan. 28, 2021

(54) SYSTEM AND METHOD FOR SIGNAL SENSING

(71) Applicant: Industrial Technology Research Institute, Hsinchu (TW)

(72) Inventors: Ting-Wu Ho, Hsinchu City (TW); Chia-Lung Liu, Hsinchu County (TW); Fu-Te Hsu, Taoyuan City (TW)

(73) Assignee: Industrial Technology Research Institute, Hsinchu (TW)

(21) Appl. No.: 16/726,948

(22) Filed: Dec. 26, 2019

Related U.S. Application Data
(60) Provisional application No. 62/876,788, filed on Jul. 22, 2019.

(30) Foreign Application Priority Data
Nov. 8, 2019 (TW) 108140738

(51) Int. Cl. A61B 5/0205; A61B 5/00; G01S 7/41

(52) U.S. Cl. CPC A61B 5/0205; A61B 5/7282; A61B 5/0816; G01S 7/415; A61B 5/7203

(57) ABSTRACT

A system and a method for signal sensing are provided. The system for signal sensing includes a processor, a transmitter, a receiver, and an oscillator. The oscillator is coupled to the transmitter and the receiver. The oscillator generates a clock signal. The transmitter transmits a plurality of output signals according to the clock signal. The receiver receives a first output signal reflected by an object according to the clock signal and obtains a channel state information according to the first output signal. The processor identifies a state of the object according to the channel state information and outputs the state of the object.

### CROSS-REFERENCE TO RELATED APPLICATION

This application claims the priority benefits of U.S. provisional application Ser. No. 62/876,788, filed on Jul. 22, 2019, and Taiwan application serial no. 108140738, filed on Nov. 8, 2019. The entirety of each of the above-mentioned patent applications is hereby incorporated by reference herein and made a part of this specification.

### BACKGROUND

### Technical Field

The disclosure relates to a system and a method for signal sensing, and more particularly to a system and a method for signal sensing based on orthogonal frequency-division multiplexing (OFDM) technology.

### Description of Related Art

Radar sensing technology is widely used in a variety of different non-contact sensing fields, for example, health care, safety monitoring, smart home, food safety enforcement, and other applications. Existing radar sensing devices (for example, doppler radar, millimeter wave (mmWave) radar, etc.) are too costly. Considering that a consumer will hesitate in making a purchase due to price considerations, using a cheap OFDM device (for example, a device using technologies such as WiFi, LTE, 5G, etc.) for non-contact sensing has been one of the most popular research techniques in recent years.

The non-contact sensing principle of an OFDM device is similar to the sonar system of a bat. The property of an object to be tested (for example, the movement of a body or the type of a liquid) causes changes in the radio wave. For example, after a transmitter transmits a signal, the signal is received by a receiver after colliding with the body of an object. Finally, the device of the receiver analyzes the received signal to identify the property of the object to be tested. However, methods mentioned above still have two key issues need to overcome.

[Issue 1: A single OFDM device is unable to sense the signal transmitted by the device itself]

Unlike existing radar sensing devices, the transmitter and receiver of the OFDM device belong to two different devices. After the device having the transmitter (also referred to as a transmitting device) transmits a signal, the device having the receiver (also referred to as a receiving device) is unable to sense the signal transmitted by the device itself. In particular, the issue of frequency offset between the transmitting device and the receiving device may lead to noise in sensing by the receiving device, causing error (for example, phase or amplitude error) in terms of signal sensing.

[Issue 2: Fresnel zone effect of an electromagnetic wave]

In general, an elliptical region with a transceiver as the focal point is formed between radio transceivers. The region is where the wireless electromagnetic wave intensity is concentrated, which accounts for about 80% of the total wireless electromagnetic wave energy. The farther the object to be tested is from the Fresnel zone, the more susceptible the sensed signal change is to be affected by the electromagnetic wave energy in the Fresnel zone.

In particular, the transmitter of the OFDM device needs to select the frequency of a subcarrier before transmitting a signal. However, in the common method, when the receiver receives the signal reflected by the object to be tested, usually only a single characteristic of the signal is used (for example, only the frequency is used or only the phase is used) for analysis to obtain relevant information of the object to be tested. However, in subcarriers of specific frequencies, the signal received by the receiver has a more significant change in amplitude but the change in phase is less obvious. It is not easy for these frequencies to be used in techniques which only use phase for analysis. In addition, in subcarriers of specific frequencies, the signal received by the receiver has a more significant change in phase but the change in amplitude is less obvious. It is not easy for these frequencies to be used in techniques which only use amplitude for analysis.

### SUMMARY

The disclosure provides a system and a method for signal sensing, which can solve the noise issue caused by frequency offset between a transmitter and a receiver, and effectively reduce the Fresnel band effect influence, thereby improving the sensing distance of an orthogonal frequency-division multiplexing (OFDM) radar.

The disclosure provides a system for signal sensing including a sensing device and a processor coupled to the sensing device. The sensing device includes a transmitter, a receiver, and an oscillator. The oscillator is coupled to the transmitter and the receiver, and is configured to generate a clock signal. The transmitter generates a plurality of subcarriers orthogonal to each other, respectively modulates a plurality of subsignals of a signal according to the plurality of subcarriers to generate a plurality of output signals, and transmits the plurality of output signals according to the clock signal. The receiver receives at least one first output signal reflected by an object in the plurality of output signals according to the clock signal and obtains a channel state information according to the first output signal. The processor identifies a state of the object according to the channel state information and outputs the state of the object.

The disclosure provides a method for signal sensing used in a system for signal sensing. The system for signal sensing includes a sensing device and a processor. The sensing device includes a transmitter, a receiver, and an oscillator coupled to the transmitter and the receiver. The method for signal sensing includes the following steps. A clock signal is generated by the oscillator. A plurality of subcarriers orthogonal to each other are generated by the transmitter, a plurality of subsignals of a signal is respectively modulated according to the plurality of subcarriers to generate a plurality of output signals, and the plurality of output signals are transmitted according to the clock signal. At least one first output signal reflected by an object in the plurality of output signals is received by the receiver according to the clock signal and a channel state information is obtained according to the first output signal. A state of the object is identified by the processor according to the channel state information and the state of the object is outputted.

Based on the above, the system and the method for signal sensing of the disclosure can integrate the transmitter and the receiver based on OFDM technology into the same device and allow the transmitter and the receiver to share the same oscillator, thereby solving the noise issue caused by the frequency offset between the transmitter and the receiver. In addition, the disclosure can also effectively reduce the Fresnel band effect influence, thereby improving the sensing distance of the OFDM radar.

### BRIEF DESCRIPTION OF THE DRAWINGS

FIG. 1 is a schematic diagram of a system for signal sensing according to an embodiment of the disclosure.

FIG. 2 is a schematic diagram of a module for signal sensing according to an embodiment of the disclosure.

FIG. 3 is a schematic diagram of a module for signal smoothing according to an embodiment of the disclosure.

FIG. 4 is a schematic diagram of a module for frequency analysis according to an embodiment of the disclosure.

FIG. 5 is a schematic diagram of a module for feature detection according to an embodiment of the disclosure.

FIG. 6 is a schematic diagram of a method for signal sensing according to an embodiment of the disclosure.

### DETAILED DESCRIPTION OF DISCLOSED EMBODIMENTS

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

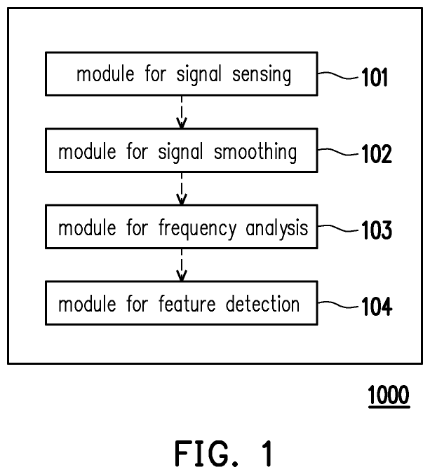

FIG. 1 is a schematic diagram of a system for signal sensing according to an embodiment of the disclosure.

Referring to FIG. 1, a system for signal sensing 1000 mainly includes a module for signal sensing 101, a module for signal smoothing 102, a module for frequency analysis 103, and a module for feature detection 104.

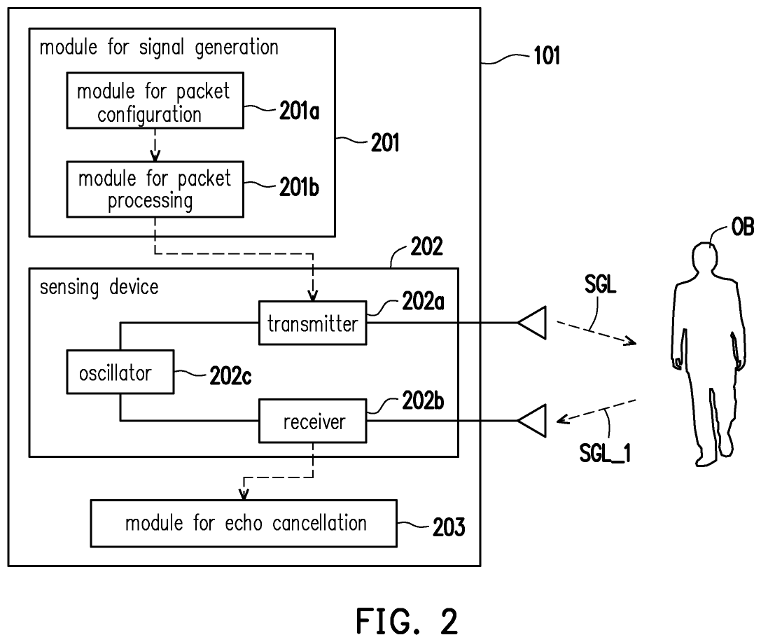

FIG. 2 is a schematic diagram of a module for signal sensing according to an embodiment of the disclosure.

Referring to FIG. 2, the module for signal sensing 101 in FIG. 1 includes a module for signal generation 201, a sensing device 202, and a module for echo cancellation 203. The module for signal generation 201 includes a module for packet configuration 201a and a module for packet processing 201b. The sensing device 202 includes a transmitter 202a, a receiver 202b, and an oscillator 202c.

In the embodiment, the system for signal sensing 1000 further includes a processor (not shown) and a storage circuit (not shown). The processor is coupled to the storage circuit and the sensing device 202. A plurality of code segments are stored in the storage circuit of the signal sensing circuit 1000. After the code segments are installed, the code segments are executed by the processor. For example, a plurality of modules are included in the storage circuit. Various operations of the module for packet configuration 201a, the module for packet processing 201b, the module for echo cancellation 203, the module for signal smoothing 102, the module for frequency analysis 103, and the module for feature detection 104 are respectively executed by the modules, wherein each module is formed by one or more code segments. However, the disclosure is not limited thereto. The various operations of the module for packet configuration 201a, the module for packet processing 201b, the module for echo cancellation 203, the module for signal smoothing 102, the module for frequency analysis 103, and the module for feature detection 104 may also be implemented by using other hardware forms.

In particular, the transmitter 202a and the receiver 202b of the disclosure may be a transceiver (or a circuit) based on orthogonal frequency-division multiplexing (OFDM) technology.

The oscillator 202c is coupled to the transmitter 202a and the receiver 202b. The oscillator 202c is configured to generate a clock signal compliant with the specifications and is simultaneously provided to the transmitter 202a and the receiver 202b as an oscillation source. In the embodiment, the transmitter 202a and the receiver 202b share the clock signal generated by the oscillator 202c.

In the embodiment, the module for signal generation 201 is configured to transmit a plurality of packets according to a packet configuration information to generate a signal. In more details, the module for packet configuration 201a in the module for signal generation 201 receives the packet configuration information set by a user or a device. The packet configuration information may be the transmission frequency of the packet. The module for packet processing 201b may, for example, cut the data to be sent into a plurality of packets according to the packet configuration information and transmit the plurality of packets to generate a signal to be transmitted through the transmitter 202a.

Then, the transmitter 202a generates a plurality of subcarriers orthogonal to each other based on OFDM operation principle, divides the signal from the module for packet processing 201b into a plurality of subsignals, and respectively modulates the plurality of subsignals according to the plurality of subcarriers to generate a plurality of output signals. Next, the transmitter 202a transmits an output signal SGL according to the packet configuration information and the clock signal of the oscillator 202c.

Thereafter, the receiver 202b receives at least one output signal SGL_1 (also referred to as a first output signal) reflected via an object OB in the output signal SGL according to the clock signal of the oscillator 202c. For example, the receiver 202b receives the output signal SGL_1 in the analog signal form according to the clock signal of the oscillator 202c and samples the output signal SGL_1 in the digital signal form.

After obtaining the output signal SGL_1, the receiver 202b obtains a channel state information according to the output signal SGL_1. The processor of the system for signal sensing 1000 identifies a state of the object OB according to the channel state information and outputs the state of the object.

In more details, in the operation of obtaining the channel state information according to the output signal SGL_1, the interference signal in the output signal SGL_1 may be first cancelled through the module for echo cancellation 203. In particular, the interference signal is transmitted via a path (also referred to as a first path) between the transmitter 202a and the receiver 202b, and the first path is not reflected via the object OB. In other words, based on the multipath issue of wireless transmission, parts of the signals transmitted by the transmitter 202a are directly transmitted from the transmitter 202a to the receiver 202b without being reflected and these signals cause error in terms of judgment. Therefore, these signals are identified as interference signals. The method of the module for echo cancellation 203 for cancelling the interference signal may be a hardware method, the multiple reference active noise control (multiple reference ANC), the recursive least squares (RLS), the least mean square (LMS), the filtered-x LMS, (FxLMS), etc.

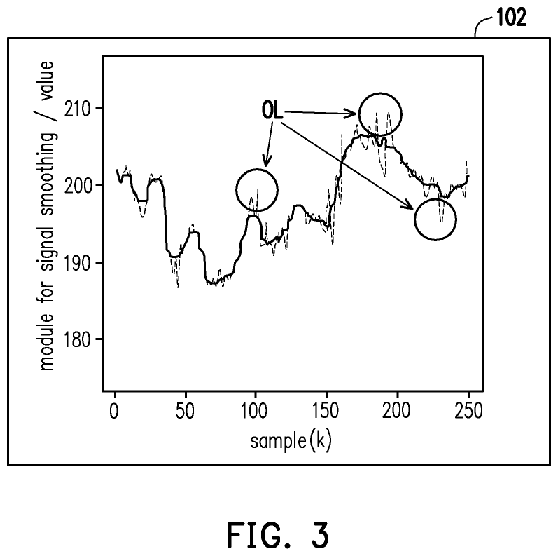

FIG. 3 is a schematic diagram of a module for signal smoothing according to an embodiment of the disclosure.

Referring to FIG. 3, after obtaining the output signal SGL_1 with the interference signal cancelled, the module for signal smoothing 102 uses a filter to filter the output signal SGL_1 with the interference signal cancelled to remove at least one outlier data OL.

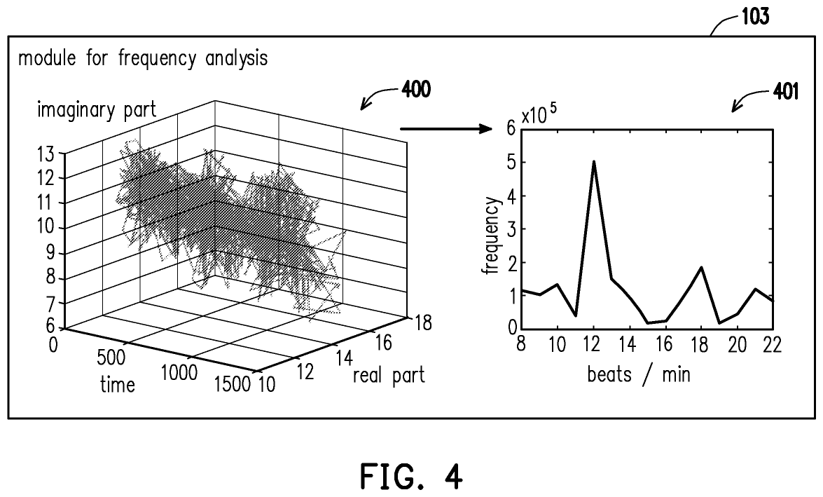

FIG. 4 is a schematic diagram of a module for frequency analysis according to an embodiment of the disclosure.

Referring to FIG. 4, after removing the interference signal and the outlier data OL in the output signal SGL_1, the module for frequency analysis 103 obtains the channel state information according to the output signal SGL_1 with the interference signal and the outlier data OL cancelled. Thereafter, the module for frequency analysis 103 obtains at least one complex number in the time domain according to the channel state information. How to obtain the channel state information according to a signal and the complex number corresponding to the channel state information can be known by conventional OFDM technology, which will not be reiterated herein. As shown by Chart 400 in FIG. 4, Chart 400 illustrates the distribution relationship of time, the real part of the complex number obtained, and the imaginary part of the complex number obtained in a three-dimensional space.

After obtaining at least one complex number in the time domain according to the channel state information, the module for frequency analysis 103 converts the complex number into a frequency domain signal in the frequency domain (as shown in Chart 401 of FIG. 4) and identifies the state of the object OB according to the frequency domain signal. In other words, the module for frequency analysis 103 describes the channel to be transmitted in the form of a complex number (for example, an IQ data) and converts the change in time of the channel to the frequency domain through a spectrum analysis method. The spectrum analysis method may be the Fourier transform (FT), the discrete wavelet transform (DWT), etc.

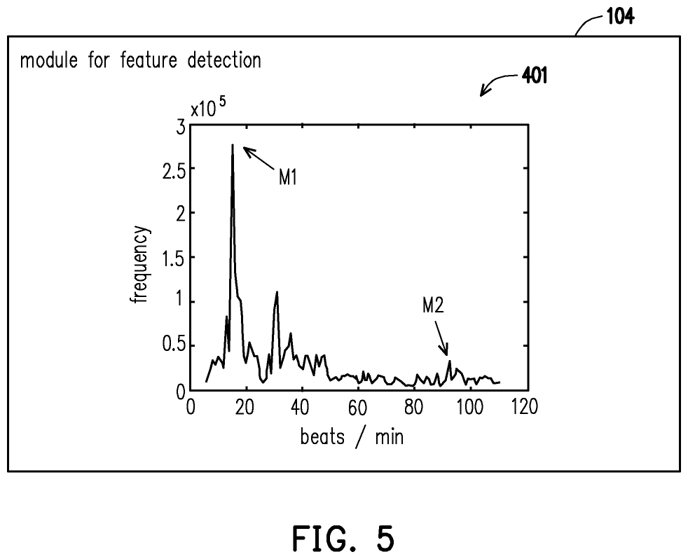

FIG. 5 is a schematic diagram of a module for feature detection according to an embodiment of the disclosure.

Referring to FIG. 5, after obtaining the frequency domain signal, the module for feature detection 104 may judge the state of the object OB. Specifically, it is assumed that the object OB is a human body and the module for feature detection 104 is configured to detect the respiratory frequency and the heartbeat frequency of the human body. Chart 401 of FIG. 5 is an example in continuation of Chart 401 of FIG. 4. In Chart 401, the dimension of the horizontal axis (also referred to as a first dimension) represents beats per minute (BPM) and the dimension of the vertical axis (also referred to as a second dimension) represents frequency. The module for feature detection 104 identifies a certain range (also referred to as a first range) in BPM (i.e. the first dimension) in the frequency domain signal and identifies a maximum value M1 (also known as a first maximum value) of frequency (i.e. the second dimension) in the first range as a first physiological information. For example, the maximum value of frequency with BPM ranging between 6 and 25 is found in the frequency domain as the respiratory frequency (i.e. the first physiological information).

Similarly, the module for feature detection 104 identifies another range (also referred to as a second range) of BPM in the frequency domain signal and identifies a maximum value M2 (also referred to as a second maximum value) of frequency in the second range as a second physiological information. For example, the maximum value of frequency with BPM ranging between 50 and 100 is found in the frequency domain as the heartbeat frequency (i.e. the second physiological information).

It should be noted that the object OB in the foregoing example is the human body and the foregoing method is configured to sense the respiratory frequency and the heartbeat frequency of the human body. However, the disclosure is not limited thereto. In an embodiment, the object OB being sensed is a liquid and the module for feature detection 104 is configured to judge the type of a liquid (for example, water or alcohol). In addition, in an embodiment, the module for feature detection 104 may also be configured to judge the position of the object OB being sensed in a space, so as to be used for positioning.

In particular, Table 1 describes the effect differences in terms of signal processing between the transmitter 202a and the receiver 202b sharing the same oscillator 202c and a conventional transmitter and receiver not sharing an oscillator.

TABLE 1

| | Respiratory | | Respiratory | |
|---|---|---|---|---|
| | frequency: 12 BPM | | frequency: 15 BPM | |
| Signal flight | Non-shared | Shared | Shared | Non-shared |
| distance | oscillator | oscillator | oscillator | oscillator |
| 2 meters | 12 | 12 | 15 | 15 |
| 4 meters | 12 | 12 | 15 | 15 |
| 6 meters | 9 | 12 | 15 | 15 |
| 8 meters | 14 | 12 | 9 | 15 |
| 10 meters | 17 | 9 | 16 | 15 |
| 12 meters | | | 13 | 15 |
| 14 meters | | | 13 | 9 |

Please refer to Table 1 above. The example in Table 1 uses amplitude for analysis. The “signal flight distance” in Table 1 represents the path length passed by the signal after being transmitted from the transmitter to be reflected by the object to the receiver. It can be clearly seen from Table 1 that when the object OB is actually breathing at 12 BPM, the device with non-shared oscillator generates an error at a signal flight distance of 6 meters while the device with shared oscillator (i.e. the system for signal sensing 1000 of the disclosure) only generates an error at a signal flight distance of 10 meters.

Similarly, when the object OB is actually breathing at 15 BPM, the device with non-shared oscillator generates an error at a signal flight distance of 8 meters while the device with shared oscillator (i.e. the system for signal sensing 1000 of the disclosure) only generates an error at a signal flight distance of 14 meters.

Table 2 describes the effect differences in terms of signal processing between the transmitter 202a and the receiver 202b sharing the same oscillator 202c and a conventional transmitter and receiver not sharing an oscillator.

TABLE 2

| | Respiratory | | Respiratory | |
|---|---|---|---|---|
| | frequency: 12 BPM | | frequency: 15 BPM | |
| Signal flight | Non-shared | Shared | Shared | Non-shared |
| distance | oscillator | oscillator | oscillator | oscillator |
| 2 meters | 12 | 12 | 15 | 15 |
| 4 meters | 12 | 12 | 15 | 15 |
| 6 meters | 9 | 12 | 15 | 15 |
| 8 meters | 14 | 12 | 9 | 15 |
| 10 meters | 17 | 12 | 16 | 15 |
| 12 meters | | | 13 | 15 |
| 14 meters | | | 13 | 16 |

Referring to Table 2 above, the device with non-shared oscillator in the example of Table 2 uses amplitude (for example, converting complex number to amplitude) for analysis while the device with shared oscillator (i.e. the system for signal sensing 1000 of the disclosure) directly observes the overall change of complex number for analysis. It can be clearly seen from Table 2 that when the object OB is actually breathing at 12 BPM, the device with non-shared oscillator generates an error at a signal flight distance of 6 meters while the device with shared oscillator (i.e. the system for signal sensing 1000 of the disclosure) still has no error at a signal flight distance of 10 meters.

Similarly, when the object OB is actually breathing at 15 BPM, the device with non-shared oscillator generates an error at a signal flight distance of 8 meters while the device with shared oscillator (i.e. the system for signal sensing 1000 of the disclosure) only generates an error at a signal flight distance of 14 meters.

As can be known from the above, the disclosure can effectively reduce the Fresnel band effect influence, thereby improving the sensing distance of the OFDM radar. In particular, the disclosure directly observes “the overall change of complex number” rather than analyzing a single characteristic by converting complex number into frequency or phase and then using one of the two as for the case of conventional technique.

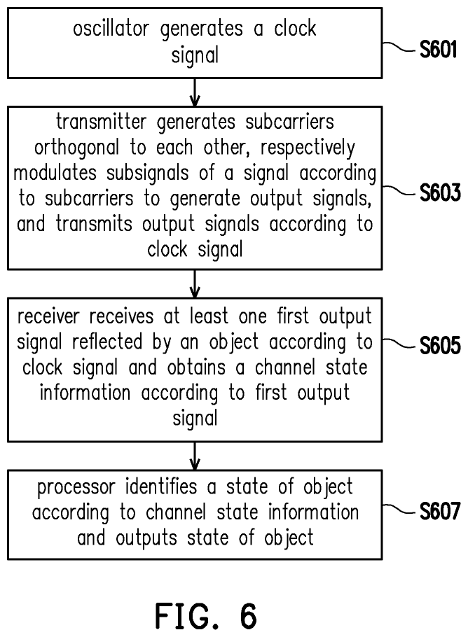

FIG. 6 is a schematic diagram of a method for signal sensing according to an embodiment of the disclosure.

Referring to FIG. 6, in Step S601, an oscillator 202c generates a clock signal. In Step S603, a transmitter 202a generates a plurality of subcarriers orthogonal to each other, respectively modulates a plurality of subsignals of a signal according to the plurality of subcarriers to generate a plurality of output signals, and transmits the plurality of output signals according to the clock signal. In Step S605, a receiver 202b receives at least one first output signal reflected by an object according to the clock signal and obtains a channel state information according to the first output signal. In Step S607, the processor identifies a state of the object according to the channel state information and outputs the state of the object.

Based on the above, the system and the method for signal sensing of the disclosure can integrate the transmitter and the receiver based on OFDM technology into the same device and allow the transmitter and the receiver to share the same oscillator, thereby solving the noise issue caused by the frequency offset between the transmitter and the receiver. In addition, the disclosure can also effectively reduce the Fresnel band effect influence, thereby improving the sensing distance of the OFDM radar.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A system for signal sensing, comprising:
a sensing device, comprising:
a transmitter;
a receiver; and
an oscillator, coupled to the transmitter and the receiver, for generating a clock signal; and
a processor for coupling to the sensing device, wherein
the transmitter generates a plurality of subcarriers orthogonal to each other, respectively modulates a plurality of subsignals of a signal according to the plurality of subcarriers to generate a plurality of output signals, and transmits the plurality of output signals according to the clock signal,
the receiver receives at least one first output signal reflected by an object in the plurality of output signals according to the clock signal and obtains a channel state information according to the first output signal, and
the processor identifies a state of the object according to the channel state information and outputs the state of the object.

2. The system for signal sensing according to claim 1, wherein in an operation of identifying the state of the object according to the channel state information,
the processor obtains at least one complex number in a time domain according to the channel state information, converts the complex number in the time domain into a frequency domain signal in a frequency domain, and identifies the state of the object according to the frequency domain signal.

3. The system for signal sensing according to claim 2, wherein the frequency domain signal comprises a first dimension and a second dimension, in an operation of identifying the state of the object according to the frequency domain signal,
the processor identifies a first range of the first dimension in the frequency domain signal and identifies a first maximum value of the second dimension in the first range as a first physiological information,
the processor identifies a second range of the first dimension in the frequency domain signal and identifies a second maximum value of the second dimension in the second range as a second physiological information.

4. The system for signal sensing according to claim 3, wherein the first dimension represents beats per minute, the second dimension represents frequency, the first physiological information represents a respiratory frequency, and the second physiological information represents a heartbeat frequency.

5. The system for signal sensing according to claim 1, further comprising:
a module for echo cancellation, wherein in an operation of obtaining the channel state information according to the first output signal,
the module for echo cancellation is configured to cancel an interference signal in the first output signal, wherein the interference signal is transmitted via a first path between the transmitter and the receiver, and the first path is not reflected via the object.

6. The system for signal sensing according to claim 5, wherein
the processor filters the first output signal with the interference signal cancelled using a filter to delete at least one outlier data.

7. The system for signal sensing according to claim 1, further comprising:
a module for signal generation for transmitting a plurality of packets according to a packet configuration information to generate the signal.

8. The system for signal sensing according to claim 1, wherein the object is a user and the state of the object is a respiratory frequency of the user.

9. The system for signal sensing according to claim 1, wherein the state of the object is a position of the object.

10. The system for signal sensing according to claim 1, wherein the object is a liquid and the state of the object is a type of the liquid.

11. A method for signal sensing used in a system for signal sensing, the system for signal sensing comprising a sensing device and a processor, the sensing device comprising a transmitter, a receiver, and an oscillator coupled to the transmitter and the receiver, the method for signal sensing comprising:
generating a clock signal by the oscillator;
generating a plurality of subcarriers orthogonal to each other, respectively modulating a plurality of subsignals of a signal according to the plurality of subcarriers to generate a plurality of output signals, and transmitting the plurality of outputs signals according to the clock signal by the transmitter;
receiving at least one first output signal reflected by an object in the plurality of output signals according to the clock signal and obtaining a channel state information according to the first output signal by the receiver; and
identifying a state of the object according to the channel state information and outputting the state of the object by the processor.

12. The method for signal sensing according to claim 11, wherein the step of identifying the state of the object according to the channel state information comprises:
obtaining at least one complex number in a time domain according to the channel state information, converting the complex number in the time domain to a frequency domain signal in a frequency domain, and identifying the state of the object according to the frequency domain signal by the processor.

13. The method for signal sensing according to claim 12, wherein the frequency domain signal comprises a first dimension and a second dimension, and the step of identifying the state of the object according to the frequency domain signal comprises:
identifying a first range of the first dimension in the frequency domain signal and identifying a first maximum value of the second dimension in the first range as a first physiological information by the processor; and
identifying a second range of the first dimension in the frequency domain signal, and identifying a second maximum value of the second dimension in the second range as a second physiological information by the processor.

14. The method for signal sensing according to claim 13, wherein the first dimension represents beats per minute, the second dimension represents frequency, the first physiological information represents a respiratory frequency, and the second physiological information represents a heartbeat frequency.

15. The method for signal sensing according to claim 11, wherein the step of obtaining the channel state information according to the first output signal comprises:
cancelling an interference signal in the first output signal by a module for echo cancellation, wherein the interference signal is transmitted via a first path between the transmitter and the receiver, and the first path is not reflected via the object.

16. The method for signal sensing according to claim 15, further comprising:
filtering the first output signal with the interference signal cancelled by the processor using a filter to delete at least one outlier data.

17. The method for signal sensing according to claim 11, further comprising:
transmitting a plurality of packets according to a packet configuration information by a module for signal generation to generate the signal.

18. The method for signal sensing according to claim 11, wherein the object is a user and the state of the object is a respiratory frequency of the user.

19. The method for signal sensing according to claim 11, wherein the state of the object is a position of the object.

20. The method for signal sensing according to claim 11, wherein the object is a liquid and the state of the object is a type of the liquid.
